(19) United States
(12) Patent Application Publication
(10) Pub. No.: US 2021/0369593 A1
(43) Pub. Date: Dec. 2, 2021

(54) Herb and Root Infused Carrier Oil

(71) Applicant: Erika Evonna Streeter Kuehner, North Hills, CA (US)

(72) Inventor: Erika Evonna Streeter Kuehner, North Hills, CA (US)

(73) Assignee: ANOVE, LLC, North Hills, CA (US)

(21) Appl. No.: 16/888,752

(22) Filed: May 31, 2020

(51) Int. Cl. A61K 8/92; A61Q 19/08; A61K 8/9794; A61K 8/9789; A61Q 19/02

(52) U.S. Cl. CPC A61K 8/922; A61Q 19/08; A61K 2800/59; A61K 8/9789; A61Q 19/02; A61K 8/9794

(57) ABSTRACT

A composition of materials for creating a hyper-effective base for various skin care, body care, and hair care formulations is disclosed. The resulting composition of materials is a superior moisturizing oil. The disclosed composition of materials provides numerous skin benefits including but not limited to improving overall skin tone and appearance, reducing inflammation and moisturizing the skin.

### CROSS REFERENCES TO RELATED APPLICATIONS

None.

### FIELD OF INVENTION

The technology relates to the general fields of skincare, personal care and cosmetics formulation and has certain specific application to oil based and anhydrous products used to improve the condition, texture, and appearance of the skin.

### STATEMENT

This substitute specification contains no new matter.

### BACKGROUND

There are many oils that can be used in skin care, body care and hair care formulations to enhance the appearance of the skin and/or hair. The composition of materials disclosed serves to describe a unique combination of ingredients which provide effective properties for addressing common skin concerns.

### SUMMARY

The disclosed oil infusion moisturizes the skin and improves the overall condition, texture and appearance of the skin. It may be used by as a base for facial and body moisturizers. Each infusion ingredient was chosen based on their proven beneficial properties and ability to address common skin concerns.

### BRIEF DESCRIPTION OF THE DRAWINGS

No drawings attached.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims. Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.
Description: The infusion is made by combining 1.5 cups (US metric measurements) dried convallaria majalis (common name lily of the valley), 1 cup dried hibiscus sabdariffa (common name hisbiscus), 2 cups dried anemopsis californica (common name yerba del manso/swamp root) and 2 cups dried paeonia alba root (common name white peony root) and 8 cups golden organic cold pressed simmondsia chinensis oil (common name jojoba oil). Once combined this mixture is left to infuse (via applied low heat or solar infusion) for 5 weeks. Solar infusion preferred. At the end of the 5 week period, oils is strained and used as a base for facial, body and hair moisturizing oils, balms, conditioners, etc.

### CONCLUSION

The disclosed embodiments are illustrative, not restrictive. While specific configurations of the material have been described, it is understood that the present invention can be applied to a wide variety of skin care, body care and hair care applications. There are many alternative ways of implementing the invention.

What is claimed is:

1. Due to the specific combination of ingredients chosen, this infused oil offers the following, superior benefits to the skin:
a. moisturizing
b. softening
c. reducing redness and inflammation
d. evening skin tone with continual use
e. improving overall skin appearance

2. This infused oil is enhanced by the following properties from each infused ingredient:
a. convallaria majalis aids in scar & blemish reduction and skin brightening
b. hibiscus sabdariffa aids in collagen synthesis due to high vitamin-c content as well as reduced collagen and elastin degradation (which prevents sagging skin and reduces the appearance of wrinkles) due to myricetin content
c. anemopsis californica provides anti-inflammatory benefits as well as skin tightening due to it's astringent properties
d. paeonia alba root aids in the fading of dark spots, skin brightening, toning and anti-aging (due to it's ability to boost collagen production and repair damaged skin cells)
e. simmondsia chinensis oil serves as the carrier oil and offers effective skin moisturization as it is similar to the skin's natural sebum and therefore easily absorbed. This oil also aids in skin repair and reducing redness

3. the described oil infusion can be used to enhance formulations across skin care, body care and hair care applications
